(19) United States
(12) Patent Application Publication
(10) Pub. No.: US 2025/0144584 A1
(43) Pub. Date: May 8, 2025

(54) APPARATUS, SYSTEM, AND METHOD FOR TRANSPORTING AND INSTALLING A FLEXIBLE BIOPROCESSING VESSEL

(71) Applicant: Pall Technology UK Limited, Portsmouth (GB)

(72) Inventors: Bart Heeren, Brussel (BE); Jorrit Sevenants, Hoegaarden (BE)

(73) Assignee: Pall Technology UK Limited, Portsmouth (GB)

(21) Appl. No.: 18/504,583

(22) Filed: Nov. 8, 2023

(51) Int. Cl. B01F 35/513; B01F 101/44; C12M 1/00

(52) U.S. Cl. CPC B01F 35/513; C12M 23/26; B01F 2101/44

(57) ABSTRACT

An apparatus for transporting and installing a flexible bioprocessing vessel includes a base portion having a front end and a rear end, the base portion configured for attachment to a hexagonal bottom surface of the flexible bioprocessing vessel. The apparatus further includes a first wing portion and a second wing portion both hingedly attached to the base portion and selectively movable between a first position and a second position. In the first position, the first wing portion and the second wing portion are angled relative to the base portion to form a storage cavity for the flexible bioprocessing vessel. In the second position, the first wing portion, the second wing portion, and the base portion are substantially planar. The apparatus has a hexagonal outer profile configured for attachment to the hexagonal bottom surface of the flexible bioprocessing vessel and for placement within the rigid hexagonal support structure.

### CROSS REFERENCE TO RELATED APPLICATIONS

The present patent application is related to concurrently filed, copending, and commonly assigned U.S. Patent Application No. entitled “FLEXIBLE BIOPROCESSING VESSEL AND RIGID SUPPORT STRUCTURE” (Attorney Docket No.: P2023-3886-US01), the disclosure of which is incorporated herein by reference.

### BACKGROUND

### Technical Field

Embodiments of the invention relate generally to bioprocessing, and, more particularly, to an apparatus, system, and method that facilitates the transportation and installation of a flexible bioprocessing vessel within a rigid support structure.

### Discussion of Art

Mixers and bioreactors are often employed to carry out biochemical and biological processes and/or manipulate liquids and other products of such processes. These devices typically utilize single-use vessels e.g., flexible or collapsible bags that are supported by an outer rigid structure such as a stainless-steel housing/tank.

In use, a disposable/single-use bag is positioned within the rigid tank and filled with the desired fluid for processing. An impeller assembly that includes a rotating impeller having one or more blades is disposed within the bag and is used to mix the fluid. Existing impeller systems are either top-driven, having a shaft that extends downwardly into the bag, on which one or more impellers are mounted, or bottom-driven, having an impeller disposed in the bottom of the bag that is driven by, for example, a magnetic drive system positioned outside the bag.

Tanks may be relatively large, having capacities of 2000 L-3000 L or more. As will be appreciated, such tanks are often installed in existing cleanroom spaces and, to that end, should be compatible with existing architecture. For example, a 3000 L tank should be able to pass through a double door having a height of 213.4 cm (7 ft) and a width of 182.9 cm (6 ft). Many cylindrical and cuboid tanks having a capacity greater than 2000 L, however, have a height that makes such passage impossible. As a result, hexagonal support structures/tanks have recently been developed that provide effective mixing at height/width ratios that allow for installation without having to modify existing architecture.

As will be appreciated, such hexagonal tanks utilize hexagonal flexible single-use bags which must be shipped and installed in the hexagonal tank. 2000 L and 3000 L flexible bags are relatively large and given their size and flexible nature, may be challenging to transport and install. Packaging solutions have been developed to assist in the transport of cuboid bags. These generally include a base plate that supports the flexible bag during transport and include handles that allow users to lift and place the bag in a tank. These solutions are not compatible with hexagonal bags and tanks, however, and utilize multiple separate components that must be secured together to form a transport structure.

Moreover, known packaging solutions (and tanks) do not include features that facilitate alignment of a flexible vessel within a rigid support structure/tank. Given the relatively unwieldy size of some flexible vessels, alignment and installation may be challenging.

In view of the above, there is a need for an apparatus, system, and method for transporting and installing a flexible bioprocessing vessel in a rigid support structure with a hexagonal interior. There's also a need for a unitary transport apparatus that includes alignment features to facilitate installation of a flexible bioprocessing vessel within a rigid support structure.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

According to an aspect of the invention, an apparatus for transporting and installing a flexible bioprocessing vessel includes a base portion having a front end and a rear end, the base portion configured for attachment to a hexagonal bottom surface of the flexible bioprocessing vessel. The apparatus further includes a first wing portion and a second wing portion, each being hingedly attached to the base portion, the first wing portion and the second wing portion are selectively movable relative to the base portion between a first position and a second position. In the first position, the first wing portion and the second wing portion are angled relative to the base portion to form a storage cavity for the flexible bioprocessing vessel that facilitates transportation and/or installation of the flexible bioprocessing vessel. In the second position, the first wing portion, the second wing portion, and the base portion are substantially planar facilitating installation and/or use of the flexible bioprocessing vessel within a rigid hexagonal support structure. The apparatus has a hexagonal outer profile that is configured for attachment to the hexagonal bottom surface of the flexible bioprocessing vessel and for placement within the rigid hexagonal support structure.

In an embodiment, the apparatus is configured for use with a hexagonal flexible bioprocessing vessel having a liquid level height to width ratio of ≤1.

In an embodiment, the apparatus, in the second position, the base portion, first wing portion and second wing portion may have a width of approximately 149 cm when configured for use with a hexagonal 2000 L flexible bioprocessing vessel.

In an embodiment, in the second position, the base portion, first wing portion, and second wing portion have a width of approximately 174 cm when configured for use with a hexagonal 3000 L flexible bioprocessing vessel.

In an embodiment, in the first position, the apparatus includes at least one locator tab that is configured to engage an alignment aperture in a bottom surface of the rigid hexagonal support structure facilitating alignment of the flexible bioprocessing vessel within the rigid hexagonal support structure.

In an embodiment, at least one of the first wing portion and the second wing portion includes a side flap that, in the first position, extends between the first wing portion and the second wing portion to define an upper surface of storage cavity.

In an embodiment, at least one of the first wing portion and the second wing portion includes a front flap and a rear flap, that, in the first position, define a front surface and rear surface of the storage cavity, respectively.

In an embodiment, the front flap includes at least one foot portion configured to fit into a slot in a bottom surface of the rigid hexagonal support structure, the slot located behind a sill of the rigid hexagonal support structure.

In an embodiment, the front flap may include an opening that facilitates connection of one or more fluid lines and/or probes to the flexible bioprocessing vessel.

In an embodiment, the at least one of the first wing portion and second wing portion includes a handle.

In an embodiment, the hexagonal bottom surface of the flexible bioprocessing vessel may extend beyond the rear end of the base portion when the apparatus is in the second position.

In an embodiment, the apparatus may also include at least one selectively removable tray configured for connection to an exterior of the base portion of the apparatus to facilitate transportation of the flexible bioprocessing vessel.

According to another aspect of the invention, a system for transporting and installing a flexible bioprocessing vessel includes a flexible bioprocessing vessel having a hexagonal body with an interior cavity configured to receive fluids for bioprocessing, the hexagonal body having a hexagonal bottom surface. The system further including a base portion attached to the hexagonal bottom surface of the flexible bioprocessing vessel, the base portion having a first wing portion and a second wing portion, each being hingedly attached to the base portion. The first wing portion and the second wing portion are selectively movable relative to the base portion between a first position and a second position. In the first position, the first wing portion and the second wing portion are angled relative to the base portion to form a storage cavity for the flexible bioprocessing vessel that facilitates transportation and/or installation of the flexible bioprocessing vessel. In the second position, the first wing portion, the second wing portion, and the base portion are substantially planar facilitating installation and/or use of the flexible bioprocessing vessel within a rigid hexagonal support structure. The base portion has a hexagonal outer profile that is configured for attachment to the hexagonal bottom surface of the flexible bioprocessing vessel and for placement within the rigid hexagonal support structure.

In an embodiment, the flexible bioprocessing vessel has a liquid level height to width ratio of ≤1.

In an embodiment, in the first position, the base portion includes at least one locator tab that is configured to engage an alignment aperture in a bottom surface of the rigid hexagonal support structure facilitating alignment of the flexible bioprocessing vessel within the rigid hexagonal support structure.

In an embodiment, at least one of the first wing portion and the second wing portion includes a side flap that, in the first position, extends between the first wing portion and the second wing portion to define an upper surface of storage cavity.

In an embodiment, at least one of the base portion, first wing portion, and/or the second wing portion includes a front flap and/or a hinged rear flap, that, in the first position, define a front surface and rear surface of the storage cavity, respectively.

In an embodiment, the front flap may include an opening that facilitates connection of one or more fluid lines and/or probes to the flexible bioprocessing vessel.

In an embodiment, the system further includes at least one selectively removable tray configured for connection to an exterior of the base portion to facilitate transportation of the flexible bioprocessing vessel.

In yet another aspect, a method for transporting a flexible bioprocessing vessel includes attaching a base portion of a support apparatus to a hexagonal bottom surface of the flexible bioprocessing vessel, the base portion having a first wing portion and a second wing portion, each being hingedly attached to the base portion, wherein at least one of the first wing portion and the second wing portion includes a side flap, a front flap and/or a hinged rear flap. The method further includes moving both the first wing portion and second wing portion relative to the base portion into a first position in which the base portion, first wing portion, second wing portion, side flap, front flap and rear flap form an interior cavity around the flexible bioprocessing vessel, the interior cavity being substantially closed on all sides and transporting the flexible bioprocessing vessel to a desired location. The base portion has a hexagonal outer profile that is configured for attachment to the hexagonal bottom surface of the flexible bioprocessing vessel and for placement within a rigid hexagonal support structure.

In an embodiment, the flexible bioprocessing vessel has a liquid level height to width ratio of ≤1.

In an embodiment, the method further includes attaching at least one selectively removable tray to an exterior of the base portion.

### BRIEF DESCRIPTION OF THE FIGURES

The present invention will be better understood from reading the

following description of non-limiting embodiments, with reference to the attached drawings, wherein below:

FIG. 1 is a perspective view of an exemplary rigid support structure suitable for use with embodiments of the present invention;

FIG. 2 is a top view of the rigid support structure of FIG. 1 depicting a hexagonal interior having a bottom surface with alignment apertures for aligning a flexible vessel placed within the hexagonal interior;

FIG. 3 is a perspective view of a flexible bioprocessing vessel having a hexagonal body suitable for use with embodiments of the invention;

FIG. 4 is a top view of an apparatus for transporting and installing a flexible bioprocessing vessel according to an embodiment of the invention;

FIG. 5 is a perspective view of the apparatus of FIG. 4 depicting the apparatus in a first position in which a first wing portion and a second wing portion of the apparatus are angled relative to a base portion to form a storage cavity for a flexible bioprocessing vessel;

FIG. 6 is a top view of an apparatus for transporting and installing a flexible bioprocessing vessel according to another embodiment of the invention;

FIG. 7 is a perspective view of the apparatus of FIG. 6 depicting the apparatus in a first position in which a first wing portion and a second wing portion of the apparatus are angled relative to a base portion to form a storage cavity for a flexible bioprocessing vessel;

FIG. 8 is a graphical illustration of users moving a flexible bioprocessing vessel utilizing an apparatus for transporting and installing a flexible bioprocessing vessel according to an embodiment of the invention;

FIG. 9 is another illustration depicting the users shown in FIG. 8 placing the apparatus and flexible bioprocessing vessel into the hexagonal interior.

FIG. 10 is another illustration depicting a locator tab on the apparatus engaging an alignment aperture.

FIG. 11 is another illustration depicting an aligned apparatus and flexible bioprocessing vessel.

FIG. 12 is another illustration depicting a user opening the aligned apparatus from FIG. 11.

FIG. 13 is another illustration of a user opening the aligned apparatus until it reaches a second position within the hexagonal interior facilitating use of the flexible bioprocessing vessel.

FIG. 14 is a perspective view of a system for transporting and installing a flexible bioprocessing vessel according to an embodiment of the invention.

FIG. 15 is a perspective sectioned view of an apparatus for transporting and installing a flexible bioprocessing vessel that includes a selectively removable tray according to an embodiment of the invention.

FIG. 16 is a side view of the selectively removable tray of FIG. 15.

FIG. 17 is a top view of an impeller seat of a vessel suitable for use with embodiments of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film.

A “vessel,” as the term is used herein, means a flexible bag, a flexible container, a semi-rigid container, or a rigid container, as the case may be. The term “vessel” as used herein is intended to encompass bioprocessing vessels having a wall or a portion of a wall that is flexible, single-use flexible bags, as well as other containers or conduits commonly used in biological or chemical processing, including, for example, cell culture/purification systems, fermentation systems, mixing systems, media/buffer preparation systems, and filtration/purification systems.

As used herein, the term “bag” means a flexible or semi-rigid vessel used, for example, as a mixer or bioreactor for the contents within.

Embodiments may be utilized in connection with a wide variety of biological and chemical processes, which are referred to generally herein as “bioprocessing.” This term encompasses, but is not limited to, the various processes that occur in bioreactors, mixers, fermenters, and the like. A “bioprocessing vessel” is a vessel suitable for use with or in a bioreactor, mixer, fermenter, or other biological or chemical processing device. Certain embodiments may be suitable for use in other industries where size, ease of installation and use, and efficient versatile mixing of fluids are desirable.

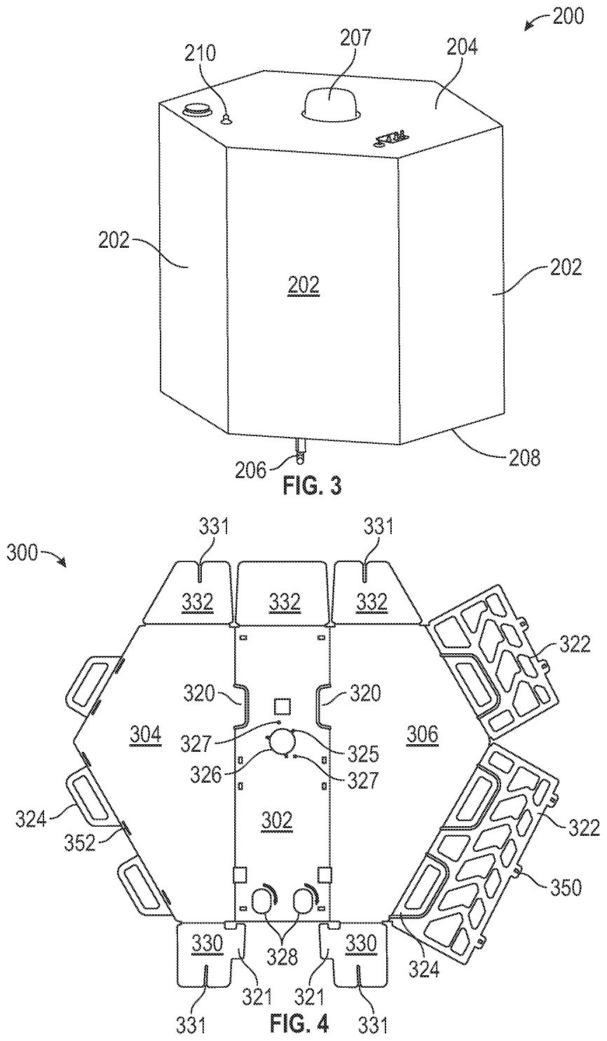

Referring to FIG. 1, a tank/rigid hexagonal support structure 100 suitable for use with embodiments of the invention is depicted. The rigid hexagonal support structure 100 includes a rigid body 102 having a hexagonal interior 104 that includes a bottom surface 106 and six sides 108 surrounding the bottom surface 106. The hexagonal interior 104 has a substantially open top 110 and a selectively openable door 112 which allows access to the hexagonal interior 104 and is configured to receive a hexagonal flexible bioprocessing vessel 200 (FIG. 3). The rigid support structure 100 further includes a stand portion 115 attached to the rigid body 102 which allows for access to the space below the body.

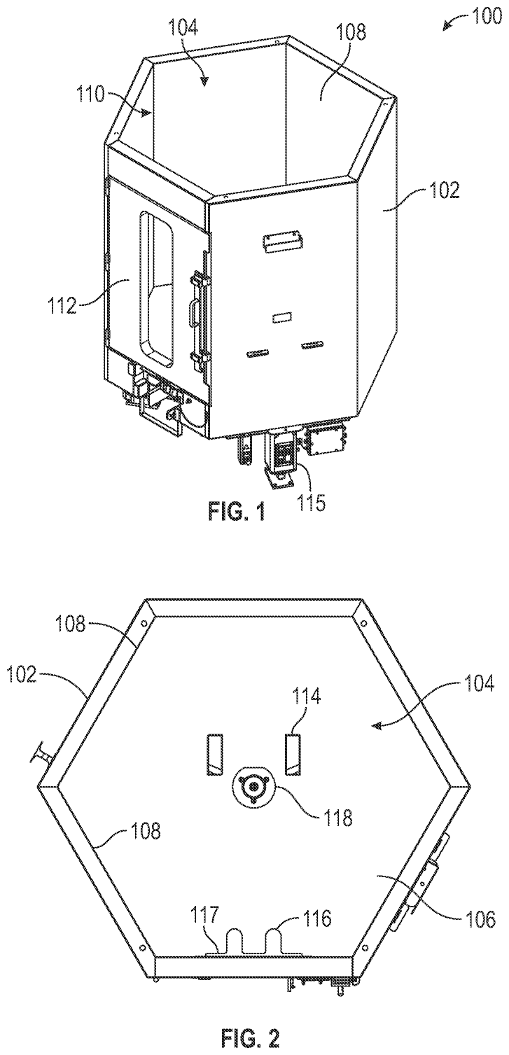

Referring to FIG. 2, the bottom surface 106 of the rigid body 102 includes at least one alignment aperture 114, which in the depicted embodiment includes two alignment apertures. The bottom surface 106 further includes openings 116 that allows for the connection of a waste line from a flexible bioprocessing vessel in the hexagonal interior 104 to an external receptacle (not shown).

Turning to FIG. 3, a hexagonal flexible bioprocessing vessel 200 configured for use with embodiments of the invention is depicted. The vessel 200 includes six flexible panels 202 forming sides of the hexagonal flexible bioprocessing vessel. The flexible bioprocessing vessel 200 also includes a top surface 204 forming a top of the hexagonal flexible bioprocessing vessel 200 and a bottom surface 208 located on an opposite end of the hexagonal flexible bioprocessing vessel 200 from the top surface 204 which forms a bottom of the hexagonal flexible bioprocessing vessel 200. The six flexible panels 202, top surface 204, and bottom surface 208 define an interior cavity configured for processing a fluid. The hexagonal flexible bioprocessing vessel 200 further includes at least one fluid input 210 and at least one fluid output 206 for adding and removing fluid to and from the interior cavity of the vessel respectively.

As mentioned, the use of hexagonal geometry as depicted in FIGS. 1-3, allows for large, e.g., 2000 L-3000 L, tanks and vessels to be installed in cleanroom spaces without altering existing interior architecture while providing effective mixing. However, existing packaging solutions to assist in shipping and installation of flexible bioprocessing vessels are not compatible with such hexagonal vessels. Known packaging solutions also utilize multiple separate components, which must be secured together to form a transport structure, and do not include alignment features that may be used with the depicted rigid hexagonal support structure 100.

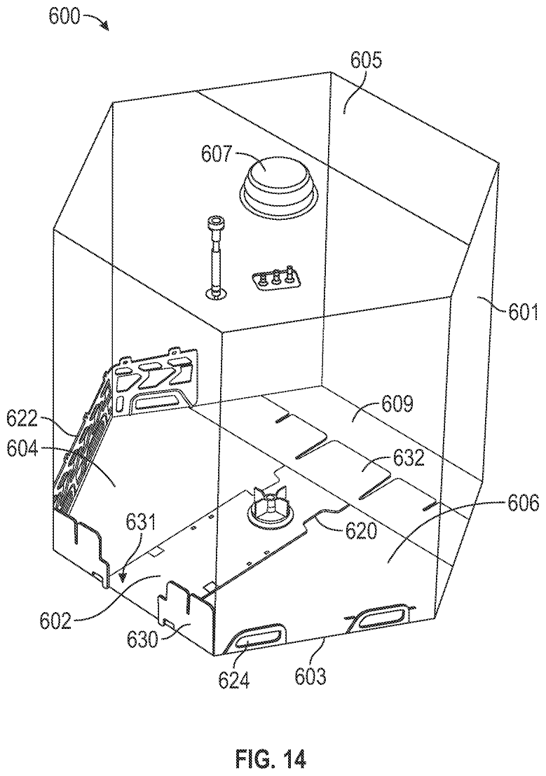

Turning now to FIGS. 4 and 5, an apparatus 300 for transporting and installing a flexible bioprocessing vessel according to an embodiment of the invention is depicted. The illustrated embodiment is configured for attachment to a 2000 L hexagonal flexible bioprocessing vessel. The apparatus 300 includes a base portion 302 having a front end/surface 360 and a rear end/surface 370. The base portion 302 is configured for attachment to a hexagonal bottom surface 609 of a flexible bioprocessing vessel (FIG. 14). The base portion 302 further includes a first wing portion 304 and a second wing portion 306, each being hingedly attached to the base portion 302. In use, the first wing portion 304 and the second wing portion 306 are selectively movable relative to the base portion 302 between a first position and a second position.

In the first position, which is depicted in FIG. 5, the first wing portion 304 and the second wing portion 306 are angled relative to the base portion 302, e.g., at an approximate 90-degree angle, to form a storage cavity 372 for a hexagonal flexible bioprocessing vessel (see e.g., FIGS. 8-13) to facilitate transportation and/or installation of the vessel.

In the second position, which is depicted in FIG. 4 and FIG. 14, the first wing portion 304, the second wing portion 306, and the base portion 302 are substantially planar facilitating installation and/or use of the flexible bioprocessing vessel within a rigid hexagonal support structure 100 (FIGS. 1 and 2).

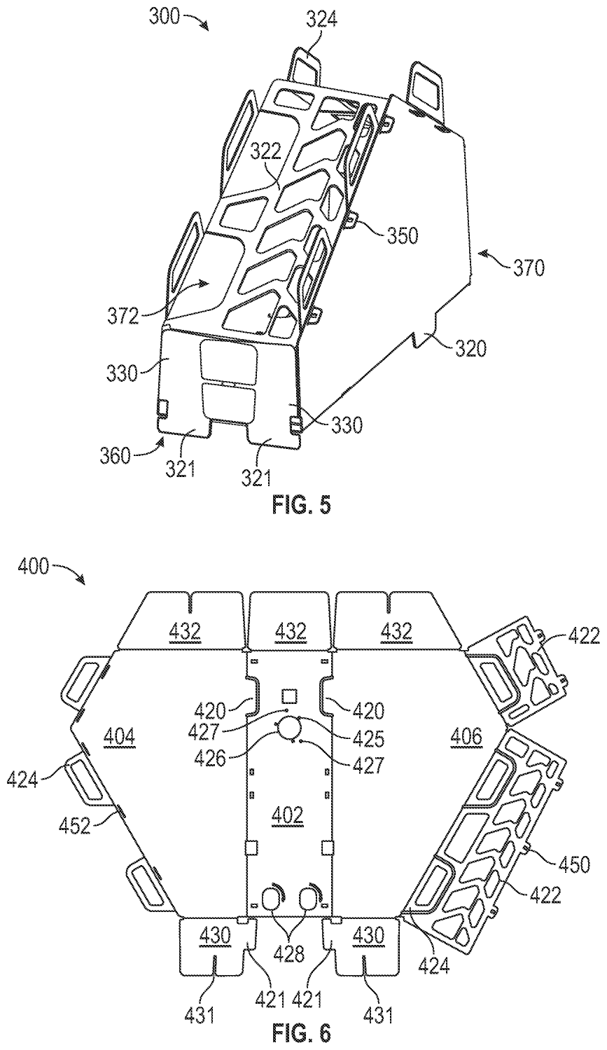

To that end, the apparatus 300 has a hexagonal outer profile that is configured for attachment to the hexagonal bottom surface 609 of the flexible bioprocessing vessel 601 and for placement within the rigid hexagonal support structure 100. While the outer profile of the apparatus is hexagonal, it need not be a regular hexagon, and, in certain embodiments, the hexagon may be irregular. An example of an apparatus 400 with an irregular hexagonal outer profile is shown in FIG. 6. In a specific alterative embodiment (not depicted), the apparatus may be substantially rectangular, but include the locating and other, non-hexagon related features described herein.

As mentioned, embodiments allow for the transportation and installation of hexagonal flexible bioprocessing vessels within rigid hexagonal support structures/tanks and specifically for hexagonal flexible bioprocessing vessels having a liquid level height to width ratio of approximately ≤1. In specific embodiments, the apparatus, (e.g., the base portion, first wing portion and second wing portion, cumulatively) has a maximum point-to-point (points of the hexagon) width of about 149 cm (edge to edge about 130 cm) when configured for use with a hexagonal 2000 L flexible bioprocessing vessel. In other embodiments, the apparatus, e.g., the base portion, first wing portion and second wing portion, cumulatively) has a maximum point-to-point width of about 174 cm (edge to edge of about 152 cm) when configured for use with a hexagonal 3000 L flexible bioprocessing vessel. As will be appreciated, however, embodiments the invention are not so limited as long as the width of the apparatus is sufficient to allow for a proper alignment and fit within a rigid hexagonal support structure.

Referring to FIG. 2, FIG. 4 and FIG. 5, the apparatus 300 includes at least one locator tab 320. In the depicted embodiment, the apparatus 300 includes two such tabs. The at least one locator tab 320 is configured to engage an alignment aperture 114 in a bottom surface 106 of the rigid hexagonal support structure 100 facilitating alignment of the flexible bioprocessing vessel within structure. In the depicted embodiment, the located tabs are formed in the first wing portion 304 and the second wing portion 306 such that when the apparatus is in the first position and the wing portions are angled upward relative to the base, the at least one tab 320 depends downward such that it may drop or fit into the alignment aperture 114 when brought into alignment with the same. As will be appreciated, in other embodiments the number and location of the at least one locator tab 320 may vary. For example, in certain embodiments, the base portion 302 may include one or more locator tabs.

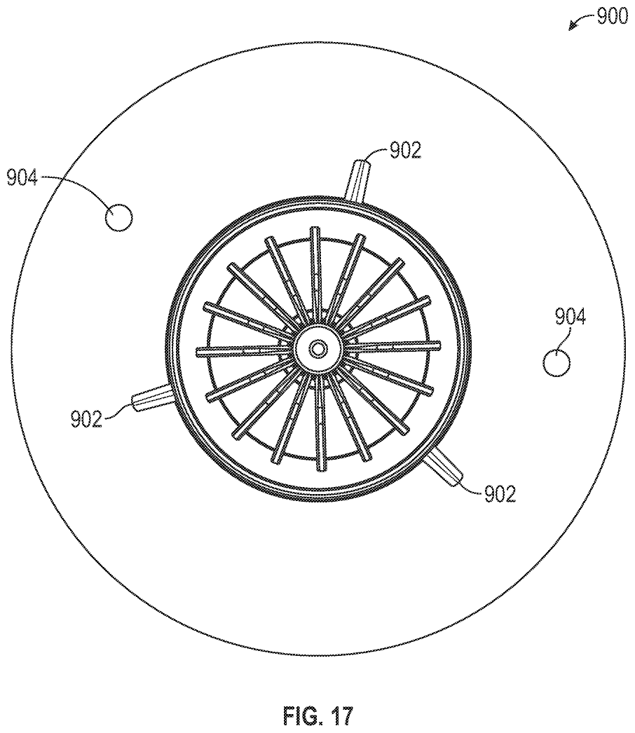

The base portion 302 further includes an opening 326 which allows the base portion 302 to be connected to a bioprocessing vessel. In the depicted embodiment, a portion of the impeller seat 900 (e.g., one or more pins 902 or protrusions thereof) external to the vessel mates with the opening 326 allowing for the apparatus 300 to be attached to the vessel (FIG. 17). In embodiments, the impeller seat 900 is inserted into the opening 326, 426 so that a series of L-shaped pins 902 on the impeller seat 900 pass through the depicted three circumferential apertures 325, 425 formed in the opening 326. 426 and then the impeller seat is rotated until fixing stubs 904 on the impeller seat snap in place into place (e.g., into corresponding openings 327, 427 in the base portion) and the pins align (e.g., with markings) on the base portion. Impeller seats suitable for use with embodiments of the invention are described in greater detail in U.S. Pat. No. 11,065,589, which is incorporated by reference in its entirety, except to the extent that any of the incorporated material is inconsistent with the express disclosure herein.

As will be appreciated, other attachment mechanisms are possible and in certain embodiments, the apparatus 300 may be thermally welded to or otherwise unitary with/permanently attached to the vessel.

In the depicted embodiment, the base portion 302 further includes two apertures 328 that allow for the connection of fluid lines such as a fluid output/waste drain line to the vessel. These fluid line apertures 328 also allow for permanently attached vessel ports to extend through the apparatus 300 during transport and installation.

In an embodiment, at least one of the first wing portion 304 and the second wing portion 306 includes a side flap 322 that, in the first position, extends between the first wing portion 304 and the second wing portion 306 to define an upper surface of storage cavity 372, as depicted in FIG. 5. Certain embodiments, such as the one depicted, a single wing portion, e.g., the second wing portion 306, may have multiple side flaps.

As shown, each side flap 322 may include tabs 350 that fit through slots 352 in the opposite wing portion. The tabs 350 may include a hole configured to accept a zip tie or like connector to fix the wing portions and side flap 322 in place forming the storage cavity 372. Of course, in other embodiments various mechanisms may be employed to secure the wing portions and side flap 322.

Each side flap 322 may include one or more integrated handles 324 that fold out when the apparatus 300 is in the first position. In any event, in embodiments, at least one of the first wing portion 304 and second wing portion 306 includes a handle 324. Here, the first wing portion 304 includes three such handles, in addition to the integrated handles 324 in each side flap 322. As will be appreciated the number and location of the handles may vary, however, ideally there are handles on either side of the apparatus 300 so that multiple users can assist in lifting, maneuvering, and installing the hexagonal flexible vessel in a rigid support structure.

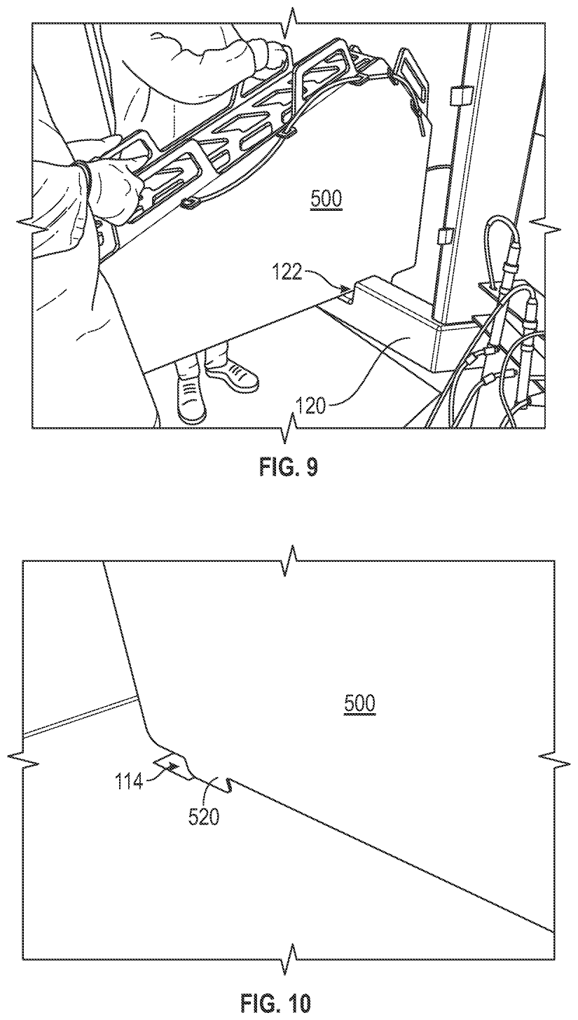

Moreover, one of the base portion 302, the first wing portion 304, and/or the second wing portion 306 includes a front flap 330 and/or a rear flap 332, that, in the first position, define a front surface 360 and/or a rear surface 370 of the storage cavity, respectively. In the depicted embodiment, each of the first wing portion and second wing portion include a front flap 330. The front flaps may be joined together when the apparatus 300 is in the first position via slots 331. As shown, in an embodiment, each of the front flaps includes a tab or foot portion 321 which also performs a locator/alignment function by dropping into slot(s) 117 behind the sill 120 of the rigid hexagonal support structure facilitating proper alignment of the vessel (FIGS. 2 and 9). The front flap(s) also includes an open or cut away portion 631 that facilitates connection of one or more fluid lines and/or probes to the flexible bioprocessing vessel 601.

In the depicted embodiment, each of the first wing portion 304, second wing portion 306, and the base portion 302 include a rear flap 332. The rear flaps 332 may be joined together, with the rear flap 332 on the base portion 302 on the inside of the other two rear flaps, when the apparatus 300 is in the first position, via slots 331.

As will be appreciated, the first and second wing portions, base portion 302, and the side, front and rear flaps, form all sides of the apparatus 300 thereby enclosing the flexible hexagonal bioprocessing vessel. In this regard, embodiments provide a unitary packaging solution.

In embodiments, the apparatus 300 may be manufactured from a rigid or semi-rigid polymeric material capable of holding the weight of the flexible hexagonal vessel without excessively deforming or breaking. In embodiments, other materials may be employed such as a compressed and/or laminated paper board. The apparatus 300 may be sterilized prior to attachment to the flexible bioprocessing vessel.

In certain embodiments, the first wing portion, second wing portion, front flap, rear flap, and handles may be hinged/foldable via a section of thinned or perforated material forming fold lines in the apparatus 300. The apparatus 300 may be manufactured via a variety of processes and the invention is not limited in this regard.

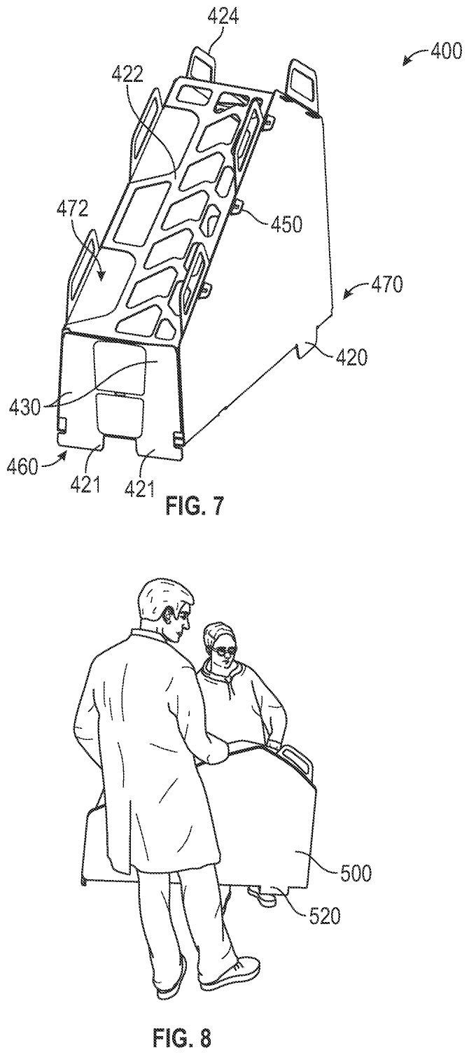

Referring now to FIG. 6 and FIG. 7, an embodiment of the inventive apparatus 400 is depicted that is configured for use with a 3000L hexagonal flexible bioprocessing vessel. This embodiment is similar to apparatus 300 with the primary differences being the width and that outer profile is that of an irregular hexagon. More specifically, the apparatus 400 includes a base portion 402 having a front end 460 and a rear end 470, a first wing portion 404, and a second wing portion 406.

The apparatus 400 includes two locator tabs 420 an opening 426 which allows the base portion to be connected to a bioprocessing vessel. The base portion 402 further includes two apertures 428 that allow for the connection of fluid lines. At least one of the first wing portion 404 and the second wing portion 406 includes a side flap 422 that, in the first position, extends between the first wing portion 404 and the second wing portion 406 to define an upper surface of storage cavity 472. Each side flap 422 includes tabs 450 that fit through slots 452 and the apparatus 400 includes multiple handles 424. Moreover, each of the first wing portion 404 and second wing portion 406 include a front flap 430 with slots 431 and rear flaps 432. In the depicted embodiment, when installed in a rigid hexagonal support structure, a hexagonal flexible vessel 601 may extend beyond the rear end of the base portion 602 and rear flaps 632 (FIG. 14).

In embodiments, in the first position, the base portion 302 is sized to fit into a notch 122 in a sill 120 that leads into the interior cavity of the rigid hexagonal support structure 100 (FIG. 9). In addition, the width of the base portion 302 may be selected so that multiple hexagonal vessels (within apparatus 300) can fit (within an external box) on a single standard size pallet. In an embodiment, a base portion 402 for a 3000 L hexagonal bioprocessing vessel has a width of about 34 cm. In an embodiment, the base portion 302 for a 2000 L vessel also has a width of about 34 cm (FIG. 4).

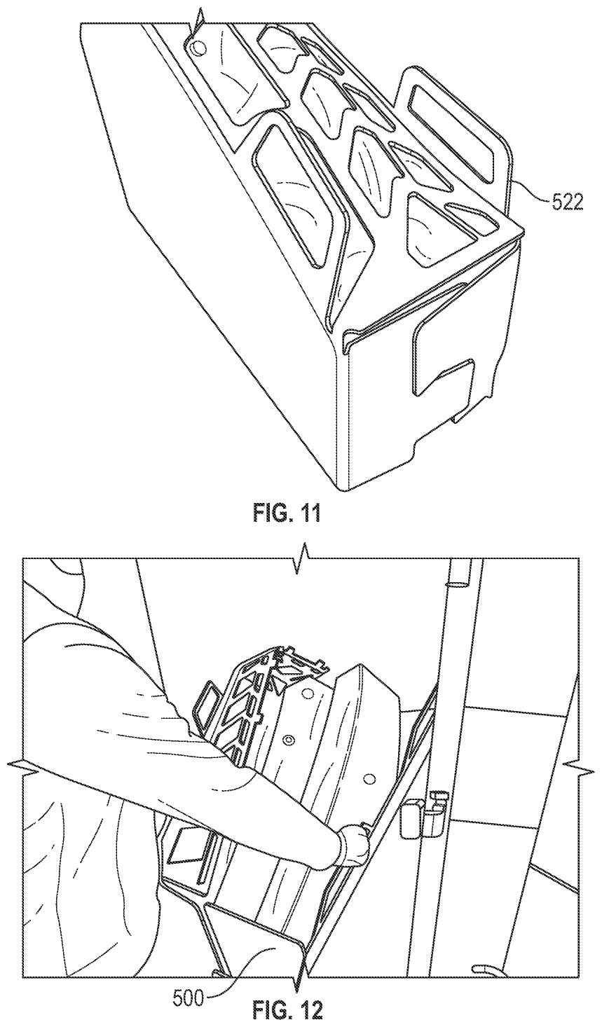

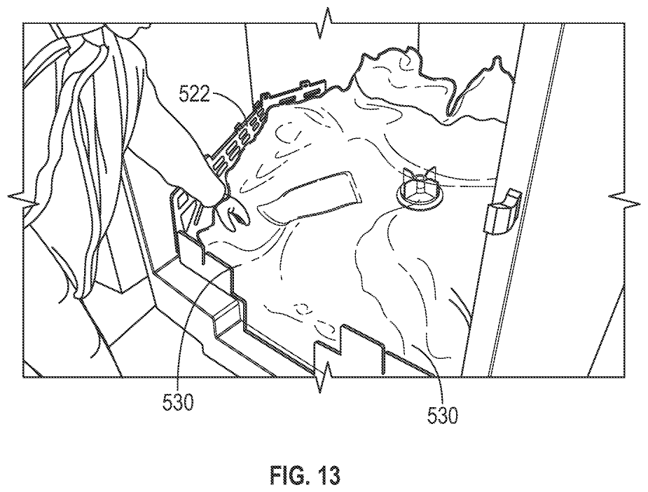

Referring now to FIG. 8, FIG. 9, FIG. 10, FIG. 11, FIG. 12, and FIG. 13, an installation method is depicted in which two users will initially lift/free the apparatus 500 (which includes the vessel in the storage cavity) from external packaging (not shown). Generally, this will initially involve two users, one on each side of the base portion gripping handles 522 located on the wing portions. The apparatus 500 is then placed on a front edge of the hexagonal interior of the rigid body of the support structure. The rigid body including a sill 120 with a notch 122 that the apparatus 500 fits into and passes through into the hexagonal interior. Once the apparatus 500 is on the sill, two users are no longer necessary and one person may continue with the installation. The locator tabs 520 are then received by the at least one alignment aperture 114, such that the apparatus 500 is in an aligned position and may be opened into the second position (FIG. 12 and FIG. 13) and installed for fluid connection and eventual use.

In an embodiment, a system 600 according to an embodiment includes a hexagonal flexible bioprocessing vessel 601 and a base portion 602 attached to the flexible bioprocessing vessel 601. The depicted embodiment shows a 3000 L hexagonal vessel and base portion 602. The base portion 602 is substantially similar to that of the apparatus shown in FIG. 6. The depicted embodiment also includes an impeller protector 607 used to cover and protect the impeller when the system is in the first position and being transported.

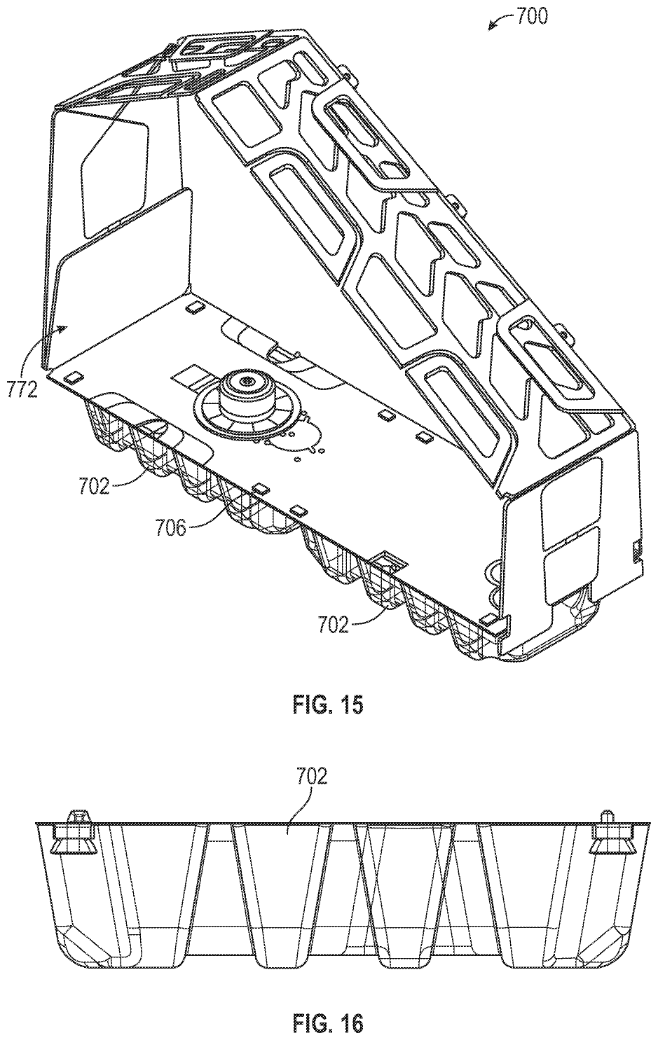

Referring now to FIG. 15 and FIG. 16, embodiments may include at least one selectively removably tray 702 configured for connection to an exterior of the base portion to protect, for example, the drain hose and/or a downwardly protruding portion of the impeller seat and provide storage during shipping while the apparatus 700 is in an external box (not shown). The selectively removable tray 702 may be secured to the base portion via tabs that fit into aperture 706, though the invention is not limited in that regard. As shown, multiple selectively removable trays may be utilized.

Returning to FIG. 14, embodiments of the invention contemplate a method for transporting a flexible bioprocessing vessel that includes attaching a base portion 602 of a support apparatus to a hexagonal bottom surface 609 of the flexible bioprocessing vessel 601, the base portion 602 having a first wing portion 604 and a second wing portion 606, each being hingedly attached to the base portion 602, wherein at least one of the first wing portion 604 and the second wing portion 606 includes a side flap 622, a front flap 630 and/or a hinged rear flap 632. The method further includes moving both the first wing portion 604 and second wing portion 606 relative to the base portion 602 into a first position in which the base portion 602, first wing portion 604, second wing portion 606, side flap 622, front flap 630 and rear flap 632 form an interior cavity around the flexible bioprocessing vessel, the interior cavity being substantially closed on all sides.

The method further involves transporting the flexible bioprocessing vessel 601 to a desired location. The base portion 602 has a hexagonal outer profile that is configured for attachment to the hexagonal bottom surface 609 of the flexible bioprocessing vessel 601 and for placement within a rigid hexagonal support structure. The method further includes attaching at least one selectively removable tray 702 to an exterior of the base portion 602.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

While the dimensions and types of materials described herein are intended to define the parameters of the invention, they are by no means limiting and are exemplary embodiments. Many other embodiments will be apparent to those of skill in the art upon reviewing the above description.

The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Moreover, in the following claims, terms such as “first,” “second,” “upper,” “lower,” “bottom,” “top,” etc. are used merely as labels, and are not intended to impose numerical or positional requirements on their objects. Further, the limitations of the following claims are not written in means-plus-function format and are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal language of the claims.

1. An apparatus for transporting and installing a flexible bioprocessing vessel comprising:
a base portion having a front end and a rear end, the base portion configured for attachment to a hexagonal bottom surface of the flexible bioprocessing vessel;
a first wing portion and a second wing portion, each being hingedly attached to the base portion;
wherein the first wing portion and the second wing portion are selectively movable relative to the base portion between a first position and a second position;
wherein in the first position, the first wing portion and the second wing portion are angled relative to the base portion to form a storage cavity for the flexible bioprocessing vessel that facilitates transportation and/or installation of the flexible bioprocessing vessel; and
wherein in the second position, the first wing portion, the second wing portion, and the base portion are substantially planar facilitating installation and/or use of the flexible bioprocessing vessel within a rigid hexagonal support structure;
wherein the apparatus has a hexagonal outer profile that is configured for attachment to the hexagonal bottom surface of the flexible bioprocessing vessel and for placement within the rigid hexagonal support structure.

2. The apparatus of claim 1, wherein the apparatus is configured for use with a hexagonal flexible bioprocessing vessel having a liquid level height to width ratio of ≤1.

3. The apparatus of claim 1, wherein, in the second position, the base portion, first wing portion and second wing portion have a width of approximately 149 cm when configured for use with a hexagonal 2000 L flexible bioprocessing vessel.

4. The apparatus of claim 1, wherein, in the second position, the base portion, first wing portion, and second wing portion have a width of approximately 174 cm when configured for use with a hexagonal 3000 L flexible bioprocessing vessel.

5. The apparatus of claim 1, wherein, in the first position, the apparatus includes at least one locator tab that is configured to engage an alignment aperture in a bottom surface of the rigid hexagonal support structure facilitating alignment of the flexible bioprocessing vessel within the rigid hexagonal support structure.

6. The apparatus of claim 1, wherein at least one of the first wing portion and the second wing portion includes a side flap that, in the first position, extends between the first wing portion and the second wing portion to define an upper surface of storage cavity.

7. The apparatus of claim 1, wherein at least one of the base portion, the first wing portion, and/or the second wing portion includes a front flap and/or a rear flap, that, in the first position, define a front surface and rear surface of the storage cavity, respectively.

8. The apparatus of claim 7, wherein the front flap includes an opening that facilitates connection of one or more fluid lines and/or probes to the flexible bioprocessing vessel.

9. The apparatus of claim 7, wherein the front flap includes at least one foot portion configured to fit into a slot in a bottom surface of the rigid hexagonal support structure, the slot located behind a sill of the rigid hexagonal support structure.

10. The apparatus of claim 1, wherein at least one of the first wing portion and second wing portion includes a handle.

11. The apparatus of claim 1, wherein the hexagonal bottom surface of the flexible bioprocessing vessel extends beyond the rear end of the base portion when the apparatus is in the second position.

12. The apparatus of claim 1 further comprising:
at least one selectively removable tray configured for connection to an exterior of the base portion of the apparatus to facilitate transportation of the flexible bioprocessing vessel.

13. A system for transporting and installing a flexible bioprocessing vessel comprising:
a flexible bioprocessing vessel comprising:
a hexagonal body having an interior cavity configured to receive fluids for bioprocessing, the hexagonal body having a hexagonal bottom surface; and
a base portion attached to the hexagonal bottom surface of the flexible bioprocessing vessel, the base portion comprising:
a first wing portion and a second wing portion, each being hingedly attached to the base portion;
wherein the first wing portion and the second wing portion are selectively movable relative to the base portion between a first position and a second position;
wherein in the first position, the first wing portion and the second wing portion are angled relative to the base portion to form a storage cavity for the flexible bioprocessing vessel that facilitates transportation and/or installation of the flexible bioprocessing vessel; and
wherein in the second position, the first wing portion, the second wing portion, and the base portion are substantially planar facilitating installation and/or use of the flexible bioprocessing vessel within a rigid hexagonal support structure;
wherein the base portion has a hexagonal outer profile that is configured for attachment to the hexagonal bottom surface of the flexible bioprocessing vessel and for placement within the rigid hexagonal support structure.

14. The system of claim 13, wherein the flexible bioprocessing vessel has a liquid level height to width ratio of ≤1.

15. The system of claim 13, wherein, in the first position, the base portion includes at least one locator tab that is configured to engage an alignment aperture in a bottom surface of the rigid hexagonal support structure facilitating alignment of the flexible bioprocessing vessel within the rigid hexagonal support structure.

16. The system of claim 13, wherein at least one of the first wing portion and the second wing portion includes a side flap that, in the first position, extends between the first wing portion and the second wing portion to define an upper surface of storage cavity.

17. The system of claim 13, wherein at least one of the first wing portion and the second wing portion includes a front flap and a hinged rear flap, that, in the first position, define a front surface and rear surface of the storage cavity, respectively.

18. The system of claim 17, wherein the front flap includes an opening that facilitates connection of one or more fluid lines and/or probes to the flexible bioprocessing vessel.

19. The system of claim 13 further comprising:
at least one selectively removable tray configured for connection to an exterior of the base portion to facilitate transportation of the flexible bioprocessing vessel.

20. A method for transporting a flexible bioprocessing vessel comprising:
attaching a base portion of a support apparatus to a hexagonal bottom surface of the flexible bioprocessing vessel, the base portion having a first wing portion and a second wing portion, each being hingedly attached to the base portion, wherein at least one of the first wing portion and the second wing portion includes a side flap, a front flap and/or a hinged rear flap;
moving both the first wing portion and second wing portion relative to the base portion into a first position in which the base portion, first wing portion, second wing portion, side flap, front flap and rear flap form an interior cavity around the flexible bioprocessing vessel, the interior cavity being substantially closed on all sides; and
transporting the flexible bioprocessing vessel to a desired location;
wherein the base portion has a hexagonal outer profile that is configured for attachment to the hexagonal bottom surface of the flexible bioprocessing vessel and for placement within a rigid hexagonal support structure.

21. The method of claim 20 wherein the flexible bioprocessing vessel has a liquid level height to width ratio of ≤1.

22. The method of claim 21 further comprising:
attaching at least one selectively removable tray to an exterior of the base portion.
